(19) United States
(12) Patent Application Publication
(10) Pub. No.: US 2014/0094787 A1
(43) Pub. Date: Apr. 3, 2014

(54) FLEXIBLE RENAL NERVE MODULATION DEVICE

(71) Applicant: Boston Scientific Scimed, Inc., (US)

(72) Inventor: BRIAN R. REYNOLDS, RAMSEY, MN (US)

(73) Assignee: BOSTON SCIENTIFIC SCIMED, INC., MAPLE GROVE, MN (US)

(21) Appl. No.: 13/631,581

(22) Filed: Sep. 28, 2012

(51) Int. Cl. A61B 18/18; A61B 18/14

(52) U.S. Cl. 606/33; 606/41

(57) ABSTRACT

Renal nerve modulation devices and methods for making and using renal nerve ablation devices are disclosed. An example renal nerve modulation device may include elongate catheter shaft having a distal portion. One or more tubular shafts may be disposed within the catheter shaft. Each of the tubular shafts may include a proximal portion, a distal portion, and a lumen extending therebetween. Each of the tubular shafts may also include a slotted portion having a plurality of slots formed therein. The slots may define a preferential zone of bending in a predetermined direction. Each of the tubular shafts may also an actuation member that is configured to shift the tubular shaft between a first configuration and a bent configuration. An ablation member may be coupled to the distal portion.

### TECHNICAL FIELD

The present disclosure pertains to medical devices, and methods for manufacturing medical devices. More particularly, the present disclosure pertains to deflectable medical devices and methods for manufacturing and using such devices.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and 15 disadvantages. There is an ongoing need to provide alternative medical devices as well as alternative methods for manufacturing and using medical devices.

### BRIEF SUMMARY

This disclosure provides design, material, manufacturing method, and use alternatives for medical devices. An example medical device may include a renal nerve modulation device. An example renal nerve modulation device may include elongate catheter shaft having a distal portion. One or more tubular shafts may be disposed within the catheter shaft. Each of the tubular shafts may include a proximal portion, a distal portion, and a lumen extending therebetween. Each of the tubular shafts may also include a slotted portion having a plurality of slots formed therein. The slots may define a preferential zone of bending in a predetermined direction. Each of the tubular shafts may also an actuation member that is configured to shift the tubular shaft between a first configuration and a bent configuration. An ablation member may be coupled to the distal portion.

Another example medical device may take the form of an assembly for renal nerve modulation. The assembly may include a catheter shaft having a proximal end portion, a distal end portion, and a lumen extending therebetween. Further, a device may be coupled to the distal portion of the catheter shaft. Here, the device may include a plurality of tubular shafts such that each tubular shaft may further include a proximal portion, distal portion, and a lumen extending therebetween. In addition, the tubular shaft may include a first slotted portion and a second slotted portion of the tubular shaft each having a plurality of slots formed therein, the first slotted portion and the second slotted portion defining a preferential zone of buckling in a predetermined direction. Further, an ablation member may be coupled to the distal portion of the tubular shaft. Still further, an actuation member may be disposed within the lumen of the tubular shaft.

An example method for treating hypertension may include providing a renal nerve modulation device. The device may include a plurality a tubular shafts. Each of the tubular shafts may include a proximal portion, a distal portion, and a lumen extending therebetween, a first slotted portion and a second slotted portion of the tubular shaft each having a plurality of slots formed therein, the first slotted portion and the second slotted portion defining a preferential zone of buckling in a predetermined direction, an ablation member coupled to the distal portion of the tubular shaft, and an actuation member disposed within the lumen of the tubular shaft. The method may also include advancing the renal nerve modulation device through the blood vessel to a position within the renal artery. Further, the method may include activating at least one ablation member.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a schematic view illustrating an example renal nerve modulation system, according to embodiments of the present disclosure.

FIG. 2A illustrates a portion of an example renal nerve modulation device.

FIG. 2B illustrates the renal nerve modulation device of FIG. 2A in deflected state.

FIG. 3 illustrates an example catheter and a renal nerve modulation device.

FIG. 4A is a side view a portion of an example renal nerve modulation device.

FIGS. 4B and 4C are a cut and flattened view of a portion of example renal nerve modulation devices.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

Certain treatments may require the temporary or permanent interruption or modification of select nerve function. One example treatment is renal nerve ablation which is sometimes used to treat conditions related to hypertension and/or congestive heart failure. The kidneys produce a sympathetic response to congestive heart failure, which, among other effects, increases the undesired retention of water and/or sodium. Ablating some of the nerves running to the kidneys may reduce or eliminate this sympathetic function, which may provide a corresponding reduction in the associated undesired symptoms.

Many nerves (and nervous tissue such as brain tissue), including renal nerves, run along the walls of or in close proximity to blood vessels and, thus, can be accessed intravascularly through the walls of the blood vessels. In some instances, it may be desirable to ablate perivascular nerves using a radio frequency (RF) electrode. In other instances, the per vascular nerves may be ablated by other means including application of thermal, ultrasonic, laser, microwave, and other related energy sources to the vessel wall.

Though the systems and methods described herein are discussed relative to hypertension therapy using a renal nerve modulation device, it is contemplated that the systems and methods may be used in other applications where renal nerve modulation is desired.

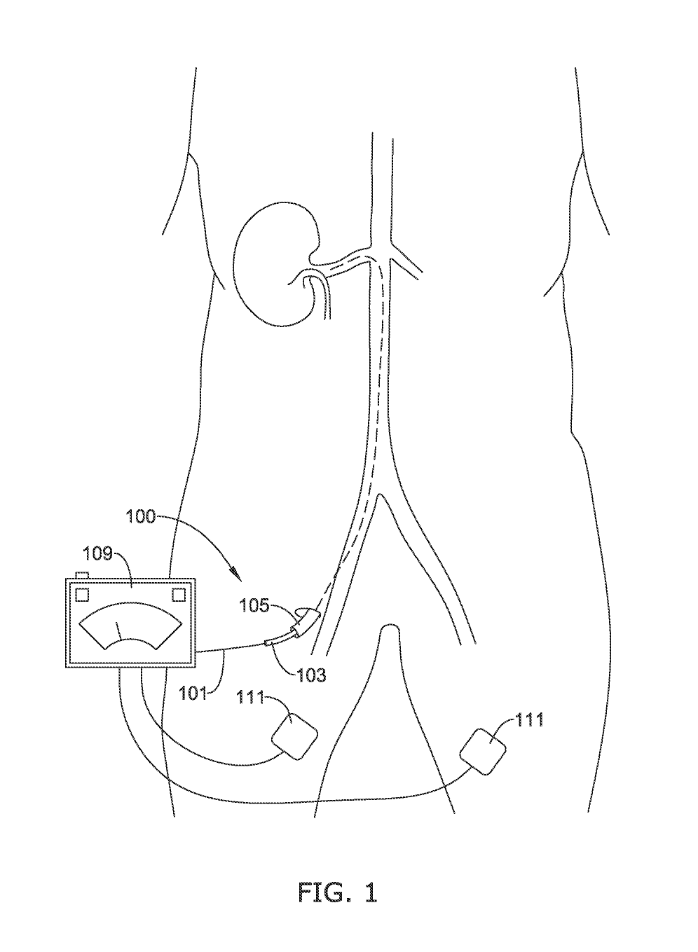

FIG. 1 is a schematic view of an illustrative renal nerve modulation system 100 in situ. System 100 may include one or more conductive element(s) 101 providing power to renal ablation device 103, which may be disposed within a catheter or sheath 105. A proximal end of conductive element 101 may be connected to a control and power element 109, which supplies the necessary electrical energy to activate the one or more electrodes at or near a distal end of the renal ablation device 103. In some instances, return electrode patches 111 may be supplied on the legs or at another conventional location on the patient's body to complete the circuit. The control and power element 109 may include monitoring elements to monitor parameters such as power, temperature, voltage, pulse size and/or shape and other suitable parameters as well as suitable controls for performing the desired procedure. The power element 109 may control a radio frequency (RF) electrode, which may be configured to operate at a frequency of approximately 460 kHz. It is contemplated that any desired frequency in the RF range may be used, for example, from 450-500 kHz. These are just examples. It is, however, contemplated that different types of energy outside the RF spectrum may be used as desired, for example, but not limited to ultrasound, microwave, and laser.

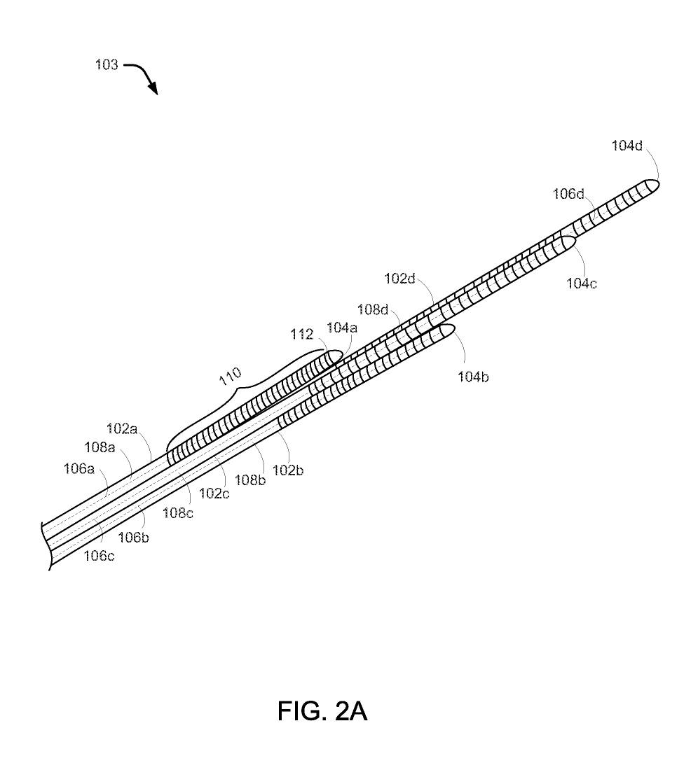

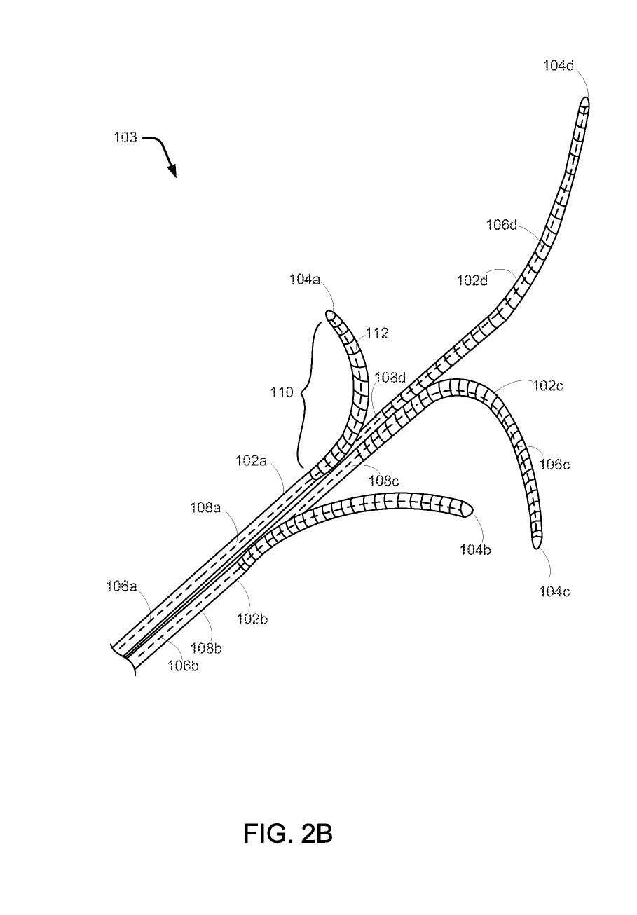

FIG. 2A illustrates an exemplary renal nerve modulation device 103, in accordance with the present disclosure. The renal nerve modulation device 103 may include a plurality of tubular shafts 102a, 102b, 102c, and, 102d (collectively, tubular shafts 102) each having a distal portion including an ablation member 104a, 104b, 104c, and 104d (collectively, ablation members 104), respectively. The proximal portion (not shown) of the tubular shafts 102 may extend proximally to a position outside the patient body. Tubular shafts 102 may further include lumens 108a, 108b, 108c, and 108d (collectively, lumens 108) extending between the proximal and the distal portions. In certain instances, the proximal portion of the tubular shafts 102 may include a hub attached thereto for connecting other diagnostic and/or treatment devices for providing a port for facilitating other interventions. In addition, the tubular shafts 102 may be disposed within (e.g., slidably disposed within) a catheter or catheter shaft (e.g., such as catheter 105 as shown in FIG. 1). In some embodiments, shafts 102 may be secured together (e.g., via welding, adhesive, thermal bond, or the like). In other embodiments, one or more of shafts 102 may be movable relative to other shafts 102.

Though the figure illustrates four tubular shafts 102, it may be contemplated that the modulation device 103 may include any suitable number of tubular shafts may be utilized for device 103, such as, but not limited to one, two, three, four, five, six, seven, eight, or more.

The use of a plurality of tubular shafts 102 may be desirable for a number of reasons. For example, because each of the tubular shafts 102 may include an ablation member 104, ablation and/or modulation may occur at a plurality of different locations along the renal artery. This may provide an overall more efficient ablation and potentially a complete or nearly complete circumferential ablation of renal nerves. In addition, because a plurality of locations may be ablated simultaneous, interventions may be completed without having to reposition the device 103. This may shorten the time of the intervention as well as provide other advantages.

As shown, each tubular shaft 102 has a tubular structure that may define a generally circular cross-section. It can be appreciated that other suitable cross-sectional shapes (e.g., including non-circular cross-sectional shapes) may also be utilized such as polygonal, irregular, or the like. In addition, the cross-section of the tubular shafts 102 may be uniform along its length or may vary along the length.

Materials employed to manufacture the tubular shafts 102 may include suitable biocompatible materials such as, but are not limited to, polymers, metals, alloys, either in combination or alone. Some example materials may include those disclosed herein. For example, tubular shafts 102 may include a nickel-titanium alloy, a nickel-chromium-molybdenum alloy, stainless steel, a non-electrically insulating polymer, combinations thereof, or the like. The material employed may have enough stiffness for use in various lumen diameters, and sufficient flexibility to maneuver through tortuous and/or stenotic lumens, avoiding any undesirable tissue injuries. To this end, the materials employed to manufacture the tubular shafts 102 may include shape memory materials such as nickel-titanium alloys.

In order to efficiently ablate the target nerves adjacent to the renal artery, it may be desirable for tubular shafts 102 to be flexible and/or otherwise configured so that the ablation members 104 may be positioned appropriately within the renal artery. This may include the use of tubular shafts and/or tubular shaft sections that have desirable bending characteristics. In addition, this may include the use of tubular shafts and/or tubular shaft sections that are deflectable or otherwise capable of being altered by a user.

Each tubular shaft 102 may include a slotted portion 110, having a plurality of slots 112 formed therein. As shown, the slotted portion 110 may be located near the distal portion of tubular shaft 102. Slots 112 may extend a desired circumferential distance around a shaft, and multiple slots may be provided on a single circumference. This slotted portion 110 may provide a bending zone, allowing tubular shaft 102 to flex or bend in a desired direction. This may include the use of a particular pattern of slots 112 that may define a preferred bending direction or orientation. Bending may occur when a control element, such as a pull wire or rod, is actuated and/or manipulated by a user. The bending direction may be dictated the disposition and spacing of slots 112. Depending on the chosen pattern, slots 112 may be formed as uniform or irregular, or they may present any other design required to achieve a desired result. Some example patterns that are contemplated for slots 112 may include those disclosed herein.

In some embodiments, slotted tubular shafts 102 may be designed to bend with a relatively low actuation force, with or without an active actuation mechanism. Collectively, these design considerations may allow tubular shaft 102 to be suited for using as a part of intervention where fine and/or tunable bending may aid the intervention. This may include renal nerve modulation (e.g., as part of a treatment for hypertension), placement of cardiac leads, other cardiac interventions, neurological interventions, gastrological interventions, or the like.

Device 103 may include ablation members 104a, 104b, 104c, and 104d (collectively, ablation member 104) coupled to the tubular shafts 102, for example at or adjacent to the distal end of the tubular shafts 102. Alternatively, the ablation members 104 may also be coupled to tubular shaft 102 at other locations such as adjacent to (but longitudinally spaced from) the distal end of the shaft 102, between slots 112, along the tubular shaft 102, or at essentially any other suitable location. For example, the ablation member 104 may be located along a curved portion of tubular shaft 102, which may provide more force between ablation member 104 and the vessel wall and/or that may aid in providing a desirable positioning/orientation relative to the vessel wall. In some embodiments, one or more ablation members 104 may be positioned away from the distal end of the tubular shafts 102. This may space the ablation members 104 from the vessel wall (e.g., providing “off-the-wall” or non-contact ablation), which may reduce damage to the vessel wall and/or provide other desirable features. These are just examples. Other suitable locations for the ablation members 104 may also be utilized.

In some embodiments, the ablation member 104 may be defined along a discrete section of the tubular shaft 102. This may include an “unslotted” section of tubular shaft 102. In other implementations, an ablation tip member may be coupled to or otherwise attached to the distal end of the tubular shaft 102.

In at least some embodiments, ablation member 104 may include a radio frequency (RF) electrode. In some of these and in other embodiments, ablation member 104 may include a thermal electrode, an ultrasound transducer, a laser electrode, a microwave electrode, combinations thereof, or the like. A suitable lead or connector (e.g., including or otherwise connected to conductive element 101) may be attached to the ablation members 104 and extend proximally therefrom (e.g., to control and power element 109). The lead may include an insulating layer or mask disposed thereon.

While each of the tubular shafts 102a, 102b, 102c, and 102d are shown with a single ablation member (e.g., ablation members 104a, 104b, 104c, and 104d), this is not intended to be limiting. Other embodiments are contemplated where one or more of the tubular shafts 102a, 102b, 102c, and 102d include a plurality of ablation members.

Further, the device 103 may include actuation members 106a, 106b, 106c, and 106d (collectively, actuation members 106) slidably disposed within the lumens 108(a-d) of the tubular shafts 102(a-d). The actuation members 106 may be adapted to impose a bending force on the tubular shafts 102.

In one embodiment, the actuation member 106 may include a pull wire, a wire rope, or the like. The actuation member 106 may be coupled to (e.g., with a weld, an adhesive, etc.) the tubular shaft 102 (for example, at or adjacent to ablation member 104, at or adjacent to slots 112, at or adjacent to a distal end of the tubular shaft 102, or the like). The actuation members 106 may extend along the exterior of the tubular shaft 102, along an interior region of the tubular shaft 102, through the wall of the tubular shafts 102, or combinations thereof to a position where it may be accessible to a clinician and can be manipulated in order to deflect the tubular shaft 102. The actuation members 106 may be utilized to bend or otherwise buckle the distal portion of the tubular shaft 102. In some embodiments, the actuation member 106 may be utilized independently from one another so that the tubular shaft 102 may be bent independently of one another. In other embodiments, the actuation member 106 may be utilized to bend multiple tubular shafts 102 (which may include bending all the tubular shafts 102) simultaneously.

In another embodiment, the actuation member 106 may also include a power element (e.g., the conductive element 101 as shown FIG. 1), which supplies the necessary energy to activate the ablation member 104 disposed near the distal portion of the tubular shaft 102. For instance, the power element may provide an electrical energy to the RF electrode disposed near the distal portion, which may thus provide energy to ablate a target tissue. In some embodiments, the actuation member 106 may be utilized independently from one another so that the ablation members 104 may be activated independently of one another. In other embodiments, the actuation member 106 may be utilized to simultaneously activate multiple ablation members 104 (which may include activating all the ablation members 104).

Materials employed to manufacture the actuation member 106 may include any suitable biocompatible having stiffness relatively larger than the material of tubular shaft 102, which thus allows the actuation member 106 to deflect and/or bend at least a portion of the tubular shaft 102. Suitable examples may include metals, alloys, polymers, or the like. In some implementations, a composite material having the desired mechanical and electrical properties may be employed. Such composites may include a combination of mechanically stable material such as, but not limited to, stainless steel and electrically conductive material such as, but not limited to, copper. Other materials are contemplated including those disclosed herein.

Once positioned adjacent the renal artery, the tubular shafts 102 may be deflected (e.g., independently or simultaneously) using the actuation members 106, as shown in FIG. 2B. Here, the distal portion of the tubular shaft 102, provided with the buckling zone of slotted portion 110, may be deflected to go different directions. In at least some embodiments, the ends of the tubular shafts 102 and/or the ablation electrodes 104 may be longitudinally separated by about 1-100 mm, or about 2-25 mm, or about 5 mm. These are just examples. Other spacing distances are contemplated. When deflected, the ends of the tubular shafts 102 and/or the ablation electrodes 104 may bend at the same angle or at different angles. Bending at different angles may desirably provide a variety of contact points where ablation electrodes 104 can approach and/or contact a vessel wall. In some embodiments, one tubular shaft (e.g., tubular shaft 102d) may be bent to an angle of 45 degrees with respect to the longitudinal axis of the tubular shafts 102. Subsequent tubular shafts may bend to a different extent with respect to the longitudinal axis. For example, tubular shaft 102c may bend an additional 45 degrees or so (e.g., 90 degrees with respect to the longitudinal axis). Tubular shaft 102a may bend in the opposite direction relative to tubular shaft 102c (e.g., “negative” 90 degrees). Tubular shaft 104b may bend in the opposite direction relative to tubular shaft 102d (e.g., “negative” 45 degrees). These are just examples. Other angles and/or configurations are contemplated.

In certain instances, the modulation device 103 may be adapted to use with a delivery device, which may facilitate introduction of the device 103 within the patient body. Exemplary delivery devices may include catheters, cannula, trocars, or other suitable devices known to those skilled in the art.

In use, device 103 may be advanced through catheter 105 to a position adjacent to an area of interest. For example, device 103 may be advanced into a renal artery. When suitable positioned, the actuation members 106 may be actuated to bend the tubular shafts 102 into the desired configuration. Energy may be supplied to the ablation member 104 to ablate and/or modulate renal nerves positioned adjacent to the renal artery.

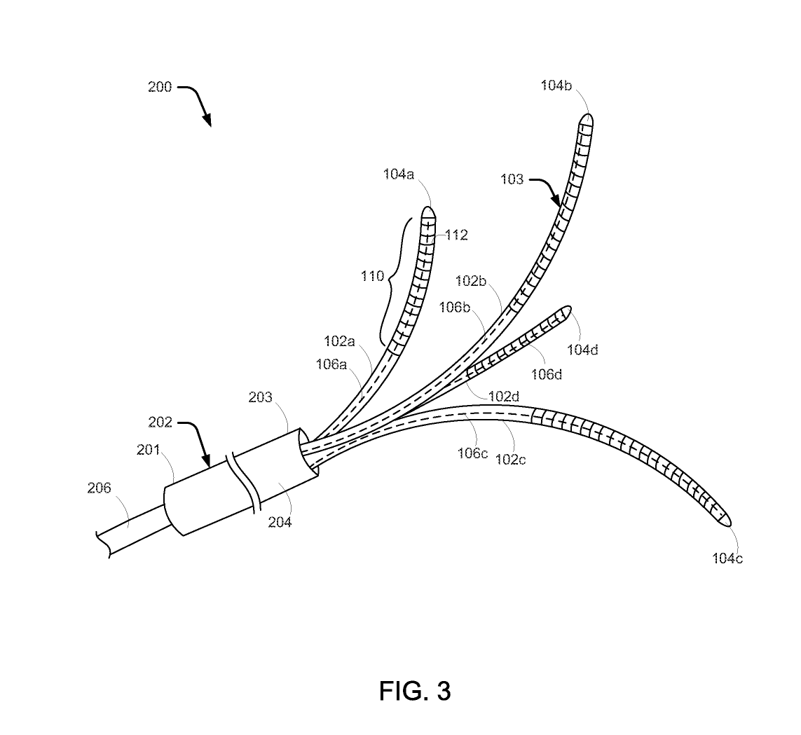

FIG. 3 illustrates a renal nerve modulation assembly or system 200 in accordance with the present disclosure. As shown, the assembly 200 may include a catheter shaft 202 with the renal nerve modulation device 103 in deflected state (as shown in FIG. 2B) attached thereto. The catheter shaft 202 may include a proximal portion 201, a distal portion 203, and a lumen 204 extending between them. The distal portion 203 may couple to the renal nerve modulation device 103. Further, the assembly 200 may include a master actuation member 206 coupled to the proximal portion 201 of the catheter shaft 202

As shown, the catheter shaft 202 has a tubular shape, which may define a circular cross-section. Other suitable cross-sections may include rectangular, polygonal, irregular, or the like. In addition, the cross-section of the catheter shafts 202 may be uniform along its length or may vary

Though not explicitly shown, the lumen 204 of the catheter shaft 202 may substantially receive the proximal portion of the tubular shafts 102. The tubular shafts 102 may pass through the entire length of the catheter shaft 202, and may eventually be attached to or otherwise coupled with the master actuation member 206. For example, the proximal portion (not shown) of the tubular shafts 102 may operably attach to the master actuation member 206. Thus, the master actuation member 206 may be adapted to simultaneously deflect and/or bend the tubular shafts 102 in a predetermined when operated by a user. In some of these and in other embodiments, the actuation member 106 may still be accessible to the clinician so that the tubular shafts 102 can be independently actuated.

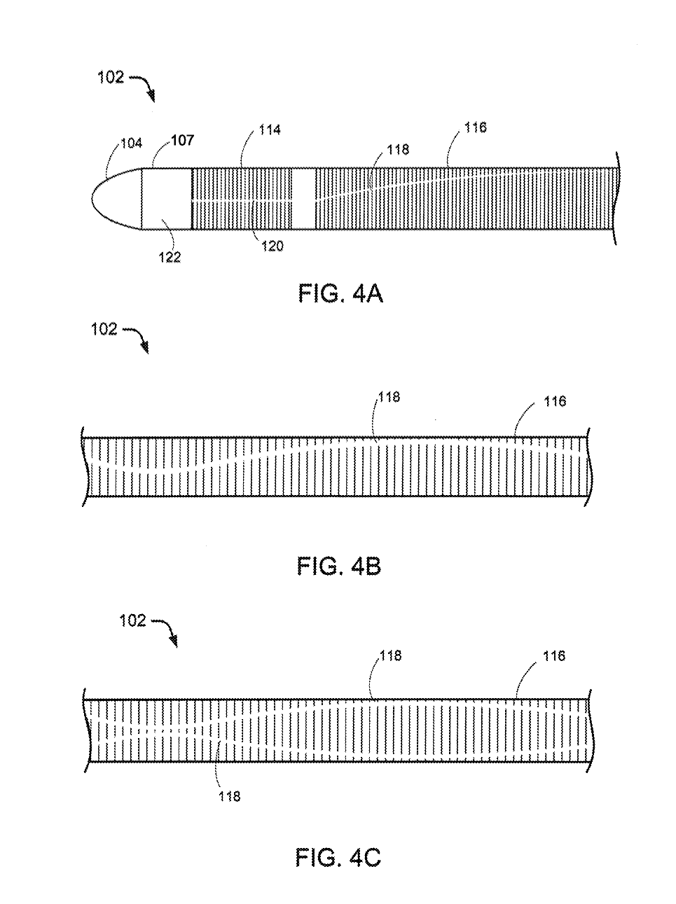

FIG. 4A is a side a portion of one example tubular shaft 102. Here, the tubular shaft 102, having a distal portion 107, may attach to an ablation member 104 through an insulating member 122 such as non-metallic section, for example.

As shown, the tubular shaft 102 may have a tubular body defining a first slotted portion 114 and second slotted portion 116 formed along the length of the tubular shaft 102. In at least some embodiments, the first and second slotted portions 114/116 are the same. Alternatively, the first and second slotted portions 114/116 may be different. For example, the first slotted portion 114 may define a longitudinally-extending beam 120 (which may also be understood as a longitudinally-extending pattern of the individual beams defined by the slots in portion 114) and the second slotted portion 116 may include another beam 118 (which may also be understood as a longitudinally-extending pattern of the individual beams defined by the slots in portion 116). Beams 118 and 120 may include portions of the tubular shafts 102 remaining after forming slots (112 of FIGS. 2A and 2B) therein.

Beams 118 and 120 may be arranged in a number of different configurations, defining different patterns. In at least some embodiments, the pattern of beams (118 and 120) may be a wave or wave-like pattern. For example, the pattern of beams 118 may be a sine wave pattern as shown in FIG. 4B. The sine-wave pattern may be derived from the general equation: y=A*sin(B*x)+C. Other patterns are contemplated including a half-sine wave pattern, a cosine wave pattern (e.g., derived from the general equation: y=A*cos(B*x)+C), a half-cosine wave pattern, other patterns based on trigonometric functions (e.g., tangent, secant, cosecant, cotangent, and/or combinations thereof), other wave patterns, non-wave or non-repetitive patterns, patterns based on mathematical functions (including exponential, polynomial, power, combinations thereof, or the like), or the like. For the purposes of this disclosure, a half-sine and half-cosine wave pattern may be understood to be a wave pattern of oscillations where only the portions of the sine/cosine wave having a positive amplitude are utilized. In other words, if a sine or cosine wave can be understood as having both peaks and valleys, a half-sine or half-cosine wave may be understood to have only the peaks. In addition to these patterns, other patterns may also be utilized and a variety of these patterns are contemplated. For examples, other oscillating patterns, squared patterns, random patterns, or other patterns may be utilized. The pattern of beam 120 may be defined by longitudinally-aligned beams extending along tubular shaft 102 where adjacent beams are (in addition to being longitudinally spaced) spatially and/or radially shifted relative to one another around tubular shaft 102 to form the pattern. Alternatively, a plurality of beams or a group (e.g., a “first” group) of longitudinally-adjacent beams may be longitudinally-aligned with one another and subsequent beams and/or groups of beams may be spatially and/or radially shifted relative to the first group of beams around tubular shaft 102 to form the pattern.

The pattern of beams 118 and 120 may desirably impact the bending characteristics of the tubular shaft 102. In at least some embodiments, the pattern of beams may be designed to bias shaft 102 to bend toward a certain direction when actuated via an actuation member (106, as shown in FIGS. 2A and 2B) or otherwise encountering an obstacle. This may include a pattern that defines a “preferred bending direction” or “single-sided deflection” configuration for tubular shaft 102. In addition, the pattern of beams 118 and 120 may define one or more discrete bending regions or bending points where bending in a desired direction occurs. For example, the pattern of beams 118 and 120 may define one, two, or more discrete bending points where tubular shaft 102 is configured to bend.

Exemplary patterns of beam 118 formed in the tubular shaft 102 may be shown in FIGS. 4B and 4C, in accordance with the present disclosure. As shown, the second slotted portion 116 may include a sine-wave like beam 118 made longitudinal the length of the tubular shaft 102. The pattern of beam 118 may be enhance the bending characteristics of the tubular shaft 102. To this end, the tubular shaft 102 may also be provided with two beams 118, as shown in FIG. 4C. This may include two sine-wave pattern beams 118. The beam patterns shown in FIGS. 4B and 4C may be utilized with any of the slotted portions disclosed herein.

The materials that can be used for the various components of system 100 (and/or other systems and/or devices disclosed herein) may include those commonly associated with medical devices. For simplicity purposes, the following discussion makes reference to device 103 and tubular member(s) 102. However, this is not intended to limit the devices and methods described herein, as the discussion may be applied to other similar tubular members and/or components of tubular members or devices disclosed herein.

Tubular shaft 102 may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also can be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of tubular shaft 102 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of device 103 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of device 103 to achieve the same result.

In some embodiments, a degree of Magnetic Resonance Imaging (MRI) compatibility is imparted into device 103. For example, tubular shaft 102 or portions thereof may be made of a material that does not substantially distort the image and create substantial artifacts (i.e., gaps in the image). Certain ferromagnetic materials, for example, may not be suitable because they may create artifacts in an MRI image. Tubular shaft 102, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

As indicated above, various embodiments of arrangements and configurations are also contemplated for slots 112 (and/or other slots 112 disclosed herein) formed in tubular shafts 102 in addition to what is described above or may be used in alternate embodiments. For example, in some embodiments, at least some, if not all of slots 112 are disposed at the same or a similar angle with respect to the longitudinal axis of tubular shaft 102. As shown, slots 112 can be disposed at an angle that is perpendicular, or substantially perpendicular, and/or can be characterized as being disposed in a plane that is normal to the longitudinal axis of tubular shaft 102. However, in other embodiments, slots 112 can be disposed at an angle that is not perpendicular, and/or can be characterized as being disposed in a plane that is not normal to the longitudinal axis of tubular shaft 102. Additionally, a group of one or more slots 112 may be disposed at different angles relative to another group of one or more slots 112.

Slots 112 may be provided to enhance the flexibility of tubular shaft 102 while still allowing for suitable torque transmission characteristics. Slots 112 may be formed such that one or more rings and/or tube segments interconnected by one or more segments and/or beams (not shown) that are formed in tubular shaft 102, and such tube segments and beams may include portions of tubular shaft 102 that remain after slots 112 are formed in the body of tubular shaft 102. Such an interconnected structure may act to maintain a relatively high degree of torsional stiffness, while maintaining a desired level of lateral flexibility. In some embodiments, some adjacent slots 112 can be formed such that they include portions that overlap with each other about the circumference of tubular shaft 102. In other embodiments, some adjacent slots 112 can be disposed such that they do not necessarily overlap with each other, but are disposed in a pattern that provides the desired degree of lateral flexibility.

Additionally, slots 112 can be arranged along the length of, or about the circumference of, tubular shaft 102 to achieve desired properties. For example, adjacent slots 112, or groups of slots 112, can be arranged in a symmetrical pattern, such as being disposed essentially equally on opposite sides about the circumference of tubular shaft 102, or can be rotated by an angle relative to each other about the axis of tubular shaft 102. Additionally, adjacent slots 112, or groups of slots 112, may be equally spaced along the length of tubular shaft 102, or can be arranged in an increasing or decreasing density pattern, or can be arranged in a non-symmetric or irregular pattern. Other characteristics, such as slot size, slot shape, and/or slot angle with respect to the longitudinal axis of tubular shaft 102, can also be varied along the length of tubular shaft 102 in order to vary the flexibility or other properties. In other embodiments, moreover, it is contemplated that the portions of the tubular member, such as a proximal section, or a distal section, or the entire tubular shaft 102, may not include any such slots 112.

As suggested herein, slots 112 may be formed in groups of two, three, four, five, or more slots 112, which may be located at substantially the same location along the axis of tubular shaft 102. Alternatively, a single slot 112 may be disposed at some or all of these locations. Within the groups of slots 112, there may be included slots 112 that are equal in size (i.e., span the same circumferential distance around tubular shaft 102). In some of these as well as other embodiments, at least some slots 112 in a group are unequal in size (i.e., span a different circumferential distance around tubular shaft 102). Longitudinally adjacent groups of slots 112 may have the same or different configurations. For example, some embodiments of tubular shaft 102 include slots 112 that are equal in size in a first group and then unequally sized in an adjacent group. It can be appreciated that in groups that have two slots 112 that are equal in size and are symmetrically disposed around the tube circumference, the centroid of the pair of beams (i.e., the portion of tubular shaft 102 remaining after slots 112 are formed therein) is coincident with the central axis of tubular shaft 102. Conversely, in groups that have two slots 112 that are unequal in size and whose centroids are directly opposed on the tube circumference, the centroid of the pair of beams can be offset from the central axis of tubular shaft 102. Some embodiments of tubular shaft 102 include only slot groups with centroids that are coincident with the central axis of the tubular shaft 102, only slot groups with centroids that are offset from the central axis of tubular shaft 102, or slot groups with centroids that are coincident with the central axis of tubular shaft 102 in a first group and offset from the central axis of tubular shaft 102 in another group. The amount of offset may vary depending on the depth (or length) of slots 112 and can include other suitable distances.

Slots 112 can be formed by methods such as micro-machining, saw-cutting (e.g., using a diamond grit embedded semiconductor dicing blade), electrical discharge machining, grinding, milling, casting, molding, chemically etching or treating, or other known methods, and the like. In some such embodiments, the structure of the tubular shaft 102 is formed by cutting and/or removing portions of the tube to form slots 112. It should be noted that the methods for manufacturing tubular shaft 102 may include forming slots 112 in tubular shaft 102 using these or other manufacturing steps. Some example embodiments of appropriate micromachining methods and other cutting methods, and structures for tubular members including slots and medical devices including tubular members are disclosed in U.S. Pat. Application Publication Nos. 2003/0069522 and 2004/0181174-A2; and U.S. Pat. Nos. 6,766,720; and 6,579,246, the entire disclosures of which are herein incorporated by reference. Some example embodiments of etching processes are described in U.S. Pat. No. 5,106,455, the entire disclosure of which is herein incorporated by reference. It should be noted that the methods for manufacturing catheter 12 may include forming slots 28 in catheter shaft 20 using these or other manufacturing steps.

In at least some embodiments, slots 112 may be formed in tubular shaft 102 using a laser cutting process. The laser cutting process may include a suitable laser and/or laser cutting apparatus. For example, the laser cutting process may utilize a fiber laser. Utilizing processes like laser cutting may be desirable for a number of reasons. For example, laser cutting processes may allow tubular shaft 102 to be cut into a number of different cutting patterns in a precisely controlled manner. This may include variations in the slot or cut width (kerf), ring width, beam height and/or width, etc. Furthermore, changes to the cutting pattern can be made without the need to replace the cutting instrument (e.g., blade). This may also allow smaller tubes (e.g., having a smaller outer diameter) to be used to form tubular shaft 102 without being limited by a minimum cutting blade size. Consequently, tubular members may be fabricated for use in neurological devices or other devices where a relatively small size may be desired. In addition, the tubular shafts 102 disclosed herein may be utilized in a guidewire (and/or as a guidewire), in a catheter (and/or as a catheter), or in other medical devices with the bending characteristics and/or deflection mechanisms as disclosed herein.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A renal nerve modulation device, comprising:
an elongate catheter shaft having a distal region; and
one or more tubular shafts disposed within the catheter shaft;
wherein each of the tubular shafts include:
a proximal portion, a distal portion, and a lumen extending therebetween,
a slotted portion having a plurality of slots formed therein, the slots defining a preferential zone of bending in a predetermined direction,
an actuation member configured to shift the tubular shaft between a first configuration and a bent configuration, and
an ablation member coupled to the distal portion.

2. The renal nerve modulation device of claim 1, wherein the ablation member is located on the actuation member.

3. The renal nerve modulation device of claim 1, wherein the ablation member is located in the distal region.

4. The renal nerve modulation device of claim 3, further comprising an insulation member, positioned to electrically isolate the ablation member.

5. The renal nerve modulation device of claim 1, wherein the tubular shaft includes a nickel-titanium alloy.

6. The renal nerve modulation device of claim 1, wherein the ablation member includes an electrode.

7. The renal nerve modulation device of claim 1, wherein the actuation member includes a pull wire.

8. The renal nerve modulation device of claim 1, wherein the actuation member is disposed within the lumen of the tubular shaft.

9. The renal nerve modulation device of claim 1, further including a master actuation member, and wherein the one or more tubular shafts are a plurality of tubular shafts, the actuation members of the tubular shafts being interconnected to the master actuation member.

10. A renal nerve modulation assembly, comprising:
a catheter shaft having a proximal end portion and a distal end portion;
a device coupled to the distal end portion of the catheter shaft, the device including a plurality of tubular shafts, each tubular shaft including:
a proximal portion, a distal portion, and a lumen extending therebetween,
a first slotted portion and a second slotted portion of the tubular shaft each having a plurality of slots formed therein, the first slotted portion and the second slotted portion defining a preferential zone of buckling in a predetermined direction,
an ablation member coupled to the distal portion of the tubular shaft, and
an actuation member disposed within the lumen of the tubular shaft.

11. The assembly of claim 10, wherein the tubular shaft includes a nickel-titanium alloy.

12. The assembly of claim 10, wherein the ablation member includes an electrode.

13. A method for treating hypertension, the method comprising:
providing a renal nerve modulation device, the renal nerve modulation device including a plurality of tubular shafts, each tubular shaft including:
a proximal portion, a distal portion, and a lumen extending therebetween,
a first slotted portion and a second slotted portion of the tubular shaft each having a plurality of slots formed therein, the first slotted portion and the second slotted portion defining a preferential zone of buckling in a predetermined direction,
an ablation member coupled to the distal portion of the tubular shaft, and
an actuation member disposed within the lumen of the tubular shaft;
advancing the renal nerve modulation device through a blood vessel to a position within a renal artery; and
activating at least one ablation member.

14. The method of claim 13, wherein advancing the renal nerve modulation device includes operating at least one actuation member to buckle at least one tubular shaft in the predetermined direction.

15. The method of claim 13, wherein activating the ablation member includes placing the ablation member in contact with a wall of the renal artery.

16. The method of claim 13, wherein activating the ablation member further includes providing an external energy source to the ablation member.

17. The method of claim 13, wherein the ablation member includes a radio frequency electrode.

18. The method of claim 13, wherein the actuation member includes a pull wire.

19. The method of claim 13, wherein at least some of the slots define a plurality of beams in the tubular shaft that are longitudinally aligned.

20. The method of claim 13, wherein the tubular shaft includes a nickel-titanium alloy.
